(19) United States
(12) Patent Application Publication
(10) Pub. No.: US 2004/0044287 A1
(43) Pub. Date: Mar. 4, 2004

(54) Identification of human tissue using optical spectroscopy

(76) Inventors: Wei-Chiang Lin, Nashville, TN (US); Anita Mahadevan-Jansen, Nashville, TN (US); E. Duco Jansen, Nashville, TN (US); Steven A. Toms, Cleveland, OR (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/360,776

(22) Filed: Apr. 22, 2002

Related U.S. Application Data
(63) Continuation-in-part of application No. 09/545,425, filed on Apr. 7, 2000, now Pat. No. 6,377,841.
(60) Provisional application No. 60/193,491, filed on Mar. 31, 2000.

(51) Int. Cl. A61B 6/00

(52) U.S. Cl. 600/475; 250/461.2; 600/477

(57) ABSTRACT

Tissue types (e.g. tumorous or normal) are determined using optical spectroscopy. Autofluorescence and diffuse reflectance spectra are generated by separately illuminating a tissue surface area with monochromatic light and white light. A peak in autofluorescence intensity (F) is provided around 460 nm from both from normal and tumorous human brain tissue with 337 nm monochromatic light excitation. Separation between white/gray matter and brain tumors is provided by certain combined F-Rd spectrum numerical values, especially certain ratios of F and Rd between 400 nm-600 nm. Numerical values based on certain combinations of unequal exponential powers of F and Rd are essentially unaffected by the superficial blood contamination. In addition, diffuse reflectance intensity (Rd) between 650 nm and 800 nm from white/gray matter was significantly stronger than that from primary and secondary brain tumors and is used with the combined spectrum numerical value to enhance accurate determinations.

### CROSS-REFERENCE TO RELATED APPLICATIONS

This application claims the benefit of U.S. Provisional Patent Application No. 60/193,491, filed Mar. 31, 2000 and is a continuation-in-part of U.S. patent application Ser. No. 09/545,425 filed Apr. 7, 2000, both entitled “Tumor Demarcation Using Optical Spectroscopy”.

### BACKGROUND OF THE INVENTION

The validity and efficacy of tissue diagnosis using optical spectroscopy, also known as optical biopsy, has been demonstrated in various tissue organs in vivo. More recently, the diagnostic capability of optical spectroscopy has been applied as a feedback tool for guidance of surgeries such as tumor resection. However, a major obstacle in the successful implementation of this idea is the inevitable presence of residual blood at the investigated tissue surface (i.e., blood contamination). This attenuates and distorts the optical signal and hence degrades the accuracy of optical spectroscopy.

In a related study on the application of optical spectroscopy (i.e., combined fluorescence and diffused reflectance spectroscopy) for brain tumor demarcation, discussed above in U.S. Application No. 60/193,491 and Ser. No. 09/545,425, it was observed that although fluorescence intensity alone can separate normal brain tissues from brain tumors with high accuracy in vitro, this was not true in vivo. This was primarily attributed to the inherent bloody nature of the operating field.

It would be a major benefit to real-time and near real-time diagnosis and treatment using optical spectroscopy if the effects of superficial blood contamination on tissue identification could be minimized. It would further be a major specific benefit to be able to identify tumorous tissue in vivo in real-time or at least near real-time while surgically removing such tumorous tissue.

### BRIEF SUMMARY OF THE INVENTION

In the broadest sense, the invention is a method for optically identifying human tissue type and comprises the steps of: illuminating a surface area of tissue to be identified with a source of white light and gathering diffuse reflectance light returned from the illuminated tissue area; illuminating the surface area of the tissue to be identified with a source of monochromatic light and gathering autofluorescent light emitted by the tissue area in response to the monochromatic light illumination, the illumination and gathering of the diffuse reflectance light and the autofluorescent light occurring in either order; generating a first ratio combination including a value of intensity of the diffuse reflectance light gathered from the illuminated tissue area and a value of intensity of the autofluorescent light gathered from the illuminated tissue area; and using the first ratio combination to identify the type of tissue of the area illuminated.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a block diagram of a tumor margin identification system of the present invention and a guided ablative laser;

FIG. 2a is an end view of a fiber optic probe;

FIG. 2b is a side elevation of the fiber optic probe end of FIG. 2a;

FIG. 2c is an illustration of the overlapping fields of view of the center fiber optic wave guide and a perimeter waive guide;

FIG. 3 if a block diagram showing the steps of data processing for tumor margin detection; and

FIG. 4 shows the theoretical attenuation effects for different simulated returned light intensity ratios;

FIG. 5 shows the measured attenuation effects for different light intensity ratios collected in vitro; and

FIG. 6 shows the variation in the blood absorption dependent coefficient “h” for different emission wavelengths and different blood standards.

### DETAILED DESCRIPTION OF THE INVENTION

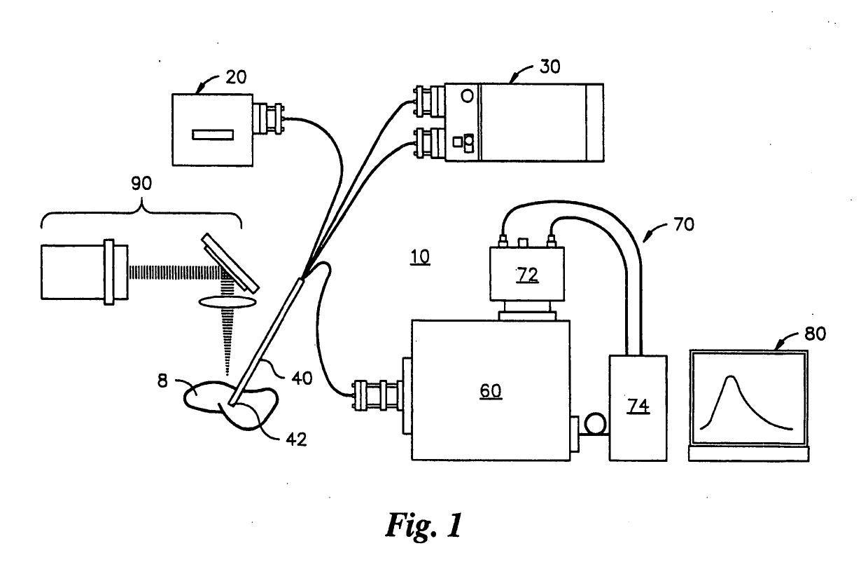

In the drawings, like numerals are used to indicate like elements throughout. The present invention relates to improvements in tissue identification using optical spectroscopy and is explained with respect to a system for tumor margin detection, specifically, in vivo brain tumor margin detection in real-time or near real time (less than one second). The components of the detection system, which is itself indicated generally at 10, are depicted in FIG. 1. They include: a source of white light 20, a source of laser light 30, a fiber optic probe 40 coupled with the source of white light 20 and the source of laser light 30 so as to deliver the white light and the laser light to a working end 42 of the probe 40; a spectrograph 60 coupled with the fiber optic probe so as to receive autofluorescent and diffuse reflectance light returned from in vivo tissue 8 contacted by the working end 42 of the probe 40 and provide a frequency spectrum of the returned light; a frequency amplitude detector 70 in the form of a CCD camera 72 with a camera controller 74, and a processor 80 in the form of a PC coupled with the spectrograph through the detector 70 and programmed to analyze the frequency spectrum of light carried from the working tip of the probe 40 to the spectrometer 60 to distinguish between light returned to the spectrograph from tumorous tissue and from non-tumorous tissue.

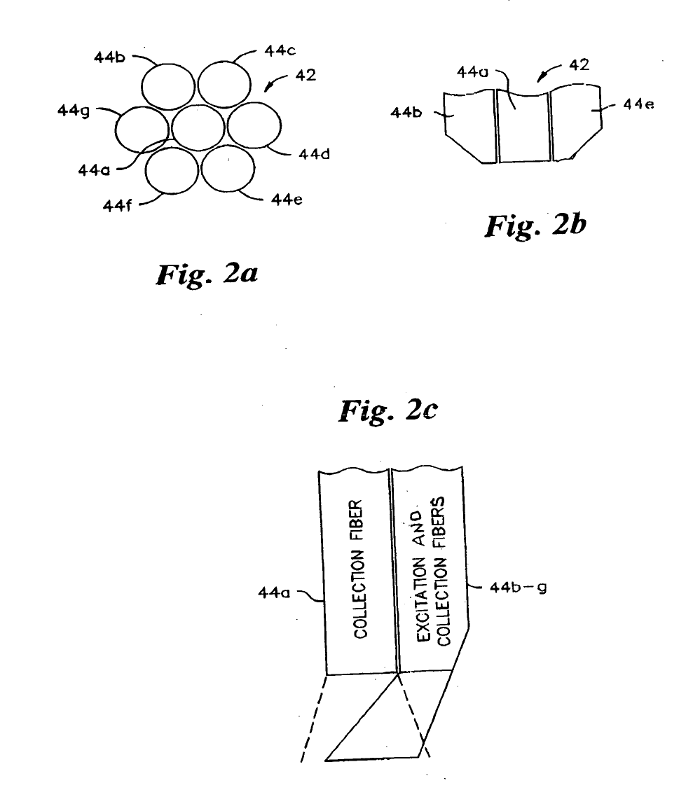

Fluorescence and diffuse reflectance spectra of tissue samples are measured with system 10 illustrated in FIG. 1. Suggestedly, a monochromatic light source (e.g. a 337 nm high-pressure nitrogen laser from Oriel Corporation, Stratford, Conn.) is used as an excitation source for autofluorescence measurements. White light source (e.g. a 150-Watt illuminator, Fiber Lite, Model 180 from Edmund Scientific Company) emitting broadband white light from 400 nm to 850 nm is used for diffuse reflectance measurements. Light delivery and collection is preferably achieved with a ‘Gaser’ fiber optic probe (Visionex, Inc., Atlanta, Ga.). This probe comprises a plurality of individual wave guides, in particular seven, in the form of 300 micron core diameter glass fibers as shown in FIGS. 2a-2c. The probe can be gas or low temperature plasma sterilized. A central fiber 44a is directed conventionally, with a squared off tip. The tips of the surrounding fibers 44b-44g are shaped, in particular tapered, to optimize overlap of excitation and collection volumes as shown in FIG. 2c. Two of the surrounding fibers, preferably diametrically opposed fibers (e.g. 44b and 44e) deliver pulses of monochromatic (laser) light and white light respectively to the tissue sample 8 (FIG. 1) while the remaining fibers 44a, 44c, 44d, 44f, 44g collect autofluorescence emission induced by the monochromatic light in and diffuse reflectance generated by the white light from the tissue sample 8. An area is preferably illuminated sequentially with the monochromatic and white light and autofluorescence and diffuse reflectance light gathered sequentially from the same illuminated area.

The gathered light is carried by the fiber optic probe 40 to the spectrograph 60 (e.g. a Triax 180 from Instruments S. A., Inc., Edison, N.J.) where it is dispersed and detected with detector 70, suggestedly a thermoelectrically cooled CCD camera (e.g. a Spectra One from Instruments S. A., Inc., Edison, N.J.). For autofluorescence measurements, reflected monochromatic (laser) light is suggestedly eliminated from the gathered light by filters, suggestedly two 360 nm long pass filters, placed in front of entrance slit of the spectrograph 60. The entire system 10 is preferably controlled by system controller 80 preferably including a processor such as a personal computer programmed to automatically take and analyze measurements and at least initially provide a tumor/not tumor output.

The system 10 is used as follows to identify tumorous tissue. The fiber optic probe 40 is placed directly in contact with the tissue sample 8 for each measurement At least three spectra are acquired by the system controller 80 at each investigated site of brain tissue sample 8: a baseline intensity level B(λ) (i.e., measured with no excitation light), a fluorescence spectrum F(λ) (measured response of the tissue sample to the monochromatic (laser) light source 30), and a reflectance spectrum Rd(λ) (measured response from the tissue sample to the white light source 20), where (λ) is the wavelength. Currently, operating lights are pointed away from the measurement site and any room lighting directly above the patient dimmed during each measurement.

The system 10 is adjusted so as to operate uniformly from measurement to measurement. The output power of the white light source is maintained at a constant maximum level, suggestedly 30 mW for the indicated fiber light source. Laser 30 is operated at a uniform repetition rate, pulse width and average pulse energy manner (suggestedly 20 Hz, 5 ns and 50±5 μJ for in vivo studies and 6.5 μJ for in vitro studies, respectively, for the above identified Oriel laser). An integration time of about 1 second or more is suggested to achieve high signal-to-noise ratio. Spectra from fluorescence and reflectance standards (i.e., F_{ref}(λ) and Rd_{ref}(λ) can be measured to monitor changes in monochromatic (laser) pulse energy, white light power, and other instrumental parameters. The fluorescence standard might be a dilute concentration of Rhodamine 6G solution (2 mg/L) in ethylene glycol contained in a quartz cuvette. The reflectance standard might be a 20% reflectance plate (e.g. a Labsphere, North Sutton, N.H.) placed in a sealed black box.

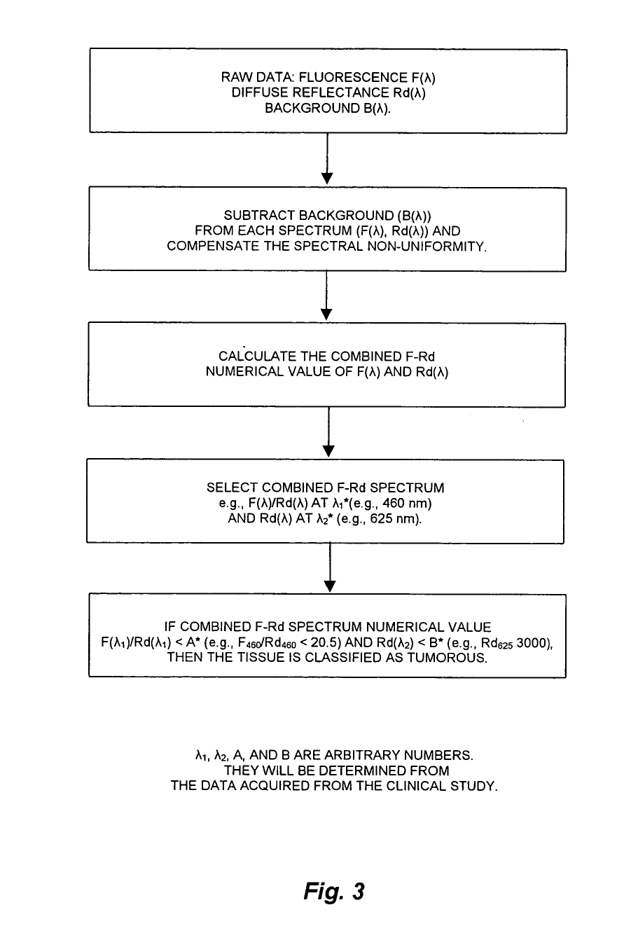

FIG. 3 depicts in diagram form the processing of the spectral data carried by the probe to the spectrograph. Spectral data is pre-processed before any analysis is conducted by the system controller 80 (e.g. the PC). Background subtraction is first performed on each selected spectrum with its corresponding baseline measurement (e.g. B (400 nm-600 nm) from F (400 nm-600 nm) and Rd (400 nm-600 nm) and B (600 nm-800 nm) from Rd (600 nm-800 nm))

Correction factors (C) are generated by taking ratios between the standard spectra (S(λ)) measured prior to the start of the study and those acquired for every experiment of the study.

Cᵢ=Sᵢ(λ)/S₁(λ) (1)

where

S(λ)=F_{ref}(λ) or Rd_{ref}(λK),

λ=620 nm for fluorescence,

700 nm for reflectance,

i=1 to n, n is the total number of experiments.

Each correction factor Cᵢ is then multiplied to every sample spectrum acquired in a given experiment i, thus ensuring spectral intensity as valid discrimination information.

All fluorescence spectra are corrected for the non-uniform spectral response of the detection system using correction factors obtained by recording the spectrum of an National Institute of Standards and Technology (NIST) traceable calibration tungsten ribbon filament lamp. Reflectance spectra are multiplied by wavelength-dependent factors to account for non-uniform spectral response of the detection system as well as spectral emission of the reflectance light source. These factors are derived from the reflectance measurement of a mirror with a known wavelength-dependent reflectivity (e.g. a 10R08ER.1 mirror from Newport Corporation, Irvine, Calif.) and are obtained before the equipment is shipped. After post-processing, changes in fluorescence and reflectance spectra, such as intensity and line shape, are correlated with histopathological identities of brain tissue sections. Empirical diagnostic algorithms are developed based on intensity, line shape, and ratio of fluorescence and diffuse reflectance spectra for separating tumorous brain tissues from normal brain tissues.

By way of background, excitation emission matrices (“EEM”) were initially measured in vitro with a standard luminescence spectrometer (Model LS 50B, Perkin-Elmer Ltd., England) on normal human brain samples (i.e., cortex) of normal and malignant brain tissues. These initial measurements showed only two distinct fluorescence peaks: one at 290 nm excitation, 350 nm (±5 nm) emission, and the other at 330 nm excitation, 460 nm (±10 nm) emission. Both fluorescence peaks were compared among the brain tissue samples. The intensity of the fluorescence peak at 330 nm excitation, 460 nm emission was found to be consistently lower in brain tumorous tissues than that in normal brain tissues. In addition, a small shift in peak location of this fluorescence emission was observed in brain tumors compared to normal brain tissue. These observations suggested that the fluorescence peak at 330 nm excitation, 460 nm emission would maximize the capability of brain tissue discrimination based on fluorescence. Therefore, a nitrogen laser (337 nm closest to 330 nm) was selected as the optimal laser excitation wavelength.

Then, fluorescence and diffuse reflectance spectra were measured using the system described in FIG. 1. Representative fluorescence and diffuse reflectance spectra were acquired from normal human brain tissues and different types of human brain tumors. In general, the fluorescence intensity at 460 nm emission of normal gray and white matter was found to be greater than that of primary and secondary tumor tissues. This observation was CE consistent with that made from EEM measurements. Diffuse reflectance of most brain tissues reached the maximum around 625 nm and then decreased gradually as wavelength increased. E Above 600 nm where blood absorption has the least influence, diffuse reflectance of white EL matter was much more intense than that of other brain tissues. However, diffuse reflectance of gray matter was similar to that of tumor tissues above 600 nm. Valleys at 415 nm, 542 nm, and 577 nm due to hemoglobin/oxyhemoglobin (Hb/HbO₂) absorption were clearly seen in fluorescence as well as diffuse reflectance spectra of brain tissues. No consistent differences, however, could be observed in the line shape of fluorescence and diffuse reflectance spectra between normal and malignant brain tissues.

Processed fluorescence and diffuse reflectance spectra from all brain tissues were analyzed in terms of intensities and ratios of intensities at different wavelengths to identify parameters that separate different brain tissue types. In addition, fluorescence spectra of all samples were normalized to their maximum to study the changes in line shape. Results of the analysis suggest different algorithms are required for separation of primary brain tumors and normal brain tissues as compared to secondary brain tumors and normal brain tissues.

A plot of fluorescence intensity at 460 nm emission (F₄₆₀) with respect to the diffuse reflectance intensity at 625 nm (Rd₆₂₅) for all normal tissues and primary tumor tissues indicated a clear separation between normal brain tissues and primary brain tumors along the F₄₆₀ axis but not along the Rd₆₂₅ axis. This indicates that fluorescence alone can differentiate normal brain tissues from primary brain tumors. Although reflectance spectra can be used to separate the samples based on white matter content, reflectance alone cannot separate between normal and tumor tissues. A simple one-dimensional discrimination algorithm, using a F₄₆₀ of 10000 calibrated units (c.u.) as the cutoff, yields a sensitivity and specificity of 97% and 96%, respectively, in separating primary brain tumors from normal brain tissues. Only two investigated sites in brain tumor samples and one in healthy gray matter were misclassified. The same discrimination algorithm was also applied to the secondary brain tumors. However, this algorithm only yielded a sensitivity of 67% in separating secondary brain tumors from normal brain tissues.

A different empirical discrimination algorithm was developed for discriminating secondary brain tumors from normal brain tissues using the ratio of fluorescence emission and diffuse reflectance at 460 nm (F₄₆₀/Rd₄₆₀) and Rd₆₂₅. A scatter plot of F₄₆₀/Rd₄₆₀ with respect to Rd₆₂₅ was generated for all normal brain tissue samples and secondary brain tumors. Using F₄₆₀/Rd₄₆₀ of 20.5 and Rd₆₂₅ of 2500 as cutoffs, this algorithm yields a sensitivity of 94% and specificity of 90% for differentiating secondary brain tumors from normal brain tissues. Only N one secondary brain tumor sample was misclassified as a normal brain tissue. The same discrimination algorithm was also applied to primary brain tumors, which yields a sensitivity of 95% and specificity of 90%. Thus, the sensitivity and specificity of this algorithm for separating all brain tumors and normal brain tissues are 96% and 90%, respectively.

In vitro studies to assess the potential of optical spectroscopy for brain tumor detection, involving spectra acquired from 127 investigated sites in brain sections from 20 patients, showed that empirical discrimination algorithms with a high specificity and sensitivity can be easily developed using fluorescence at 460 nm emission and diffuse reflectance at 460 nm and 625 nm. These results attest the validity of using combined fluorescence and diffuse reflectance spectroscopy for discrimination of primary and secondary tumors from normal brain tissues.

All fluorescence spectra acquired in the in vitro study exhibited only one fluorescence peak at 460 nm (±10 nm) emission using 337 nm excitation or longer. This observation is different from those reported previously in which multiple fluorescence peaks were measured at various excitation wavelengths. In addition, no definite change in the line shape was found between the fluorescence spectra of normal brain tissues and those of brain tumors. The fluorescence based empirical discrimination developed in this study, therefore, only utilizes the fluorescence intensity of about 460 nm emission (F₄₆₀). This discrimination algorithm performs very well in separating primary brain tumors from normal brain tissues; sensitivity of 97% and specificity of 96% are achieved. The success of this algorithm is attributed to F₄₆₀ which is consistently lower in primary brain tumors than that in normal brain tissues. However, this fluorescence based discrimination algorithm is less effective in separating secondary brain tumors from normal brain tissues due to strong F₄₆₀ from some secondary brain tumors.

To circumvent the limitation of the fluorescence based algorithm in differentiating secondary brain tumors, a second discrimination algorithm is developed based on combined fluorescence and diffuse reflectance, F₄₆₀/Rd₄₆₀ and Rd₆₂₅. The ratio of F₄₆₀ and Rd₄₆₀ is used to reduce fluorescence spectral distortion introduced by tissue reabsorption and scattering. Rd₆₂₅ is selected because of the differences in its intensity between different brain tissue types with minimum influence from absorption of Hb/HbO₂. This algorithm is effective in differentiating secondary brain tumors from normal brain tissues, with a sensitivity of 94% and specificity of 90%. It separates all brain tumors from normal brain tissue with a sensitivity and specificity of 96% and 90%. It should be noted that both algorithms were developed based on the current data set and should be considered as biased.

Tissue fluorescence intensity is determined not only by the concentration of natural fluorophores within the tissue but also by the optical properties of the tissue. Hence, interpreting changes in the fluorescence spectra of various brain tissue types is complex. It has been suggested that the concentration of many natural fluorophores, such as nicotinamide adenine dinucleotide (NADH), varies between normal and malignant tissues. In addition, increase in hemoglobin content, which leads to an increase in absorption coefficient at 337 nm as well as 460 nm, could also reduce the fluorescence intensity at 460 nm emission. While the specific cause(s) for the variations in the fluorescence intensity at 460 nm emission in the different brain tissues types is not yet known, the interdependence of tissue optics and the fluorescence emission indicates that the accuracy of a discrimination algorithm based on fluorescence intensity alone may be degraded by, for example, blood contamination.

Distinct architectural changes at the cellular and sub-cellular level are exhibited between normal and malignant brain tissues. For example, brain white matter is relatively anuclear but most aggressive tumors are characterized with a high density of cells (and therefore nuclei) and a higher nuclear-cytoplasmic ratio. Thus optical properties vary significantly between different brain tissue types. However, diffuse reflectance alone is insufficient for brain tissue discrimination as the level of diffuse reflectance from gray matter is very similar to those from brain tumors. This may seem incoherent with the optical properties measurements of brain tissues reported by others who found that the ratio of absorption and scattering coefficient from gray matter is lower than that from brain tumors, especially between 600 nm and 800 nm. However, it should be noted that the intensity of diffuse reflectance at a fixed radial position (Rd(r)) does not necessarily correlate linearly to the variations in absorption and scattering coefficients of tissue samples. Hence the same Rd(r) may be measured from two samples with different optical properties. This has been verified with a Monte Carlo simulation program.

In vivo data showed good correlation with those obtained in vitro. In particular, the F₄₆₀/Rd₄₆₀ ratio cutoff was changed to 22 and the Rd₆₂₅ cutoff changed to 3030 to yield a sensitivity and specificity of seventy-eight percent and seventy-six percent, respectively. A two-step imperical discrimination method based on the combined F-Rd spectrum numerical value F₄₆₀/Rd₄₆₀ and on Rd₆₂₅ was able to yield a sensitivity of eighty-nine percent.

The primary effect of superficial blood contamination on tissue optical spectra is that it causes additional attenuation of light at both the excitation and emission wavelengths. Assuming that the layer of blood at the tissue surface is optically thin and homogenous, the light attenuation A [%] resulting from this blood layer can be described using Beer's law as A=exp[−μₐ(λ)×d]×100%, where μₐ(λ) [cm⁻¹] is the wavelength dependent absorption coefficient of blood layer and d [cm] is the thickness of the blood layer. Here we denote the exponent (μₐ×d), as the attenuation coefficient α. The fluoresence signal F(λₘ)_{c} measured from a blood contaminated tissue sample therefore, can be written as,

$$\begin{matrix}{F ( {λ}_{m} )}_{c} = {F ( {λ}_{m} )}_{o} \times exp [ - {μ}_{a} ( {λ}_{x} ) \times d ] \times exp [ - {μ}_{a} ( {λ}_{m} ) \times d ] \\ = {F ( {λ}_{m} )}_{o} \times exp [ - ( k + 1 ) \times {μ}_{a} ( {λ}_{m} ) \times d ]\end{matrix} \qquad ( 2 )$$

where λₖ is the emission (return) wavelength, λₓ is the excitation (incident) wavelength, F(λₘ)₀ is the fluorescence intensity at λₘ from tissue without blood contamination, and k=λₐ(λₓ)/μₐ(λₘ). The same principle can also be applied to diffuse reflectance Rd(λₘ)_{c}.

$$\begin{matrix}{Rd ( {λ}_{m} )}_{c} = {Rd ( {λ}_{m} )}_{o} \times exp [ - {μ}_{a , m} \times d ] \times exp [ - {μ}_{a , m} \times d ] \\ = {Rd ( {λ}_{m} )}_{o} \times exp [ - 2 \times {μ}_{a , m} \times d ]\end{matrix} \qquad ( 3 )$$

where Rd(λₘ)ₒ is the diffuse reflectance at λₘ returned from tissue without blood contamination.

The exponential terms in equations (2) and (3), can be easily removed by taking the ratio of F(λₘ)_{c} and the h-th power of Rd(λₘ)_{c}. As a result,

F(λₘ)_{c}/Rd^{h}(λₘ)_{c}=F(λₘ)ₒ/Rd^{(k+1)/2}(λₘ)ₒ (4)

In other words, the ratios will be equal where the exponent or exponential value h=(k+1)/2.

Since hemoglobin is the primary chromophore in blood in the spectral region of interest (i.e., 300-700 nm and, more particularly, 300-600 nm) and the partial pressure of oxygen in air is about 150 mmHg, the absorption spectrum of blood at the tissue surface should be similar to that of oxyhemoglobin. In the application of optical spectroscopy for brain tumor resection guidance, the fluorescence excitation wavelength used is 337 nm and the corresponding tissue fluorescence has its primary peak around 460 nm. Thus, selecting (λₓ)=337 nm and (λₘ)=460 nm, k=2.36 and h=1.68 is obtained. Hence, the theory predicts that the numerical value of ratio F/Rd^{1.68} at 460 nm emission should be independent of the degree of superficial blood contamination.

A two-layer Monte Carlo fluorescence model was used for initial validation. The Monte Carlo fluorescence model was used to predict the distribution of reflected excitation photons Rd and remitted fluorescence photons F as a function of radial position r [cm] and escape angle Φ [degree]. The tissue phantom stimulated in the model was homogenous, semiinfinite medium consisting of two optically distinct layers: a 100 μm thick absorbing medium at the top simulating surface blood and a 5 cm thick bottom layer simulating matter in the brain. The optical properties of the two layers of the tissue phantom used in the simulations are shown in Table 1 where n is the index of reflection, μ'ₛ is the reduced scattering coefficient and B+is the blood content [%] ranging from 0% to 90% in steps of 10%.

TABLE I

| | Optical Properties | | | | | Fluorescence | |
|---|---|---|---|---|---|---|---|
| | μₐ, 337 nm | μ_{‘s}, 337 nm | μₐ, 460 nm | μ_{‘s}, 337 nm | | Efficiency | Thickness |
| Layer | [cm⁻¹] | [cm⁻¹] | [cm⁻¹] | [cm⁻¹] | n | Q | d[cm] |
| 1 | 545 × B⁺ | 0 | 231 × B⁺ | 0 | 1.4 | 0 | 0.01 |
| 2 | 4.72 | 76.7 | 2 | 67.2 | 1.4 | 0.01 | 5 |

The excitation wavelength was maintained at 337 nm and the emission wavelength at 460 nm. The excitation light used in each simulation was a collimated beam with a uniform beam profile and a beam diameter of 300 μm. Overall, ten tissue phantoms with varying degrees of blood contamination were simulated. The number of photons used in each run of simulation was equal to or greater than 500,000 to ensure the statistical accuracy of the simulation. Two data arrays F₄₆₀(r, Φ) and Rd₄₆₀(r, Φ) were generated from each run of the simulation. The values of F₄₆₀ and Rd₄₆₀ from each phantom were calculated by summing those remitted fluorescence photons and reflected excitation photons, respectively, from r=150 μm to 450 μm and Φ=0° to 30°.

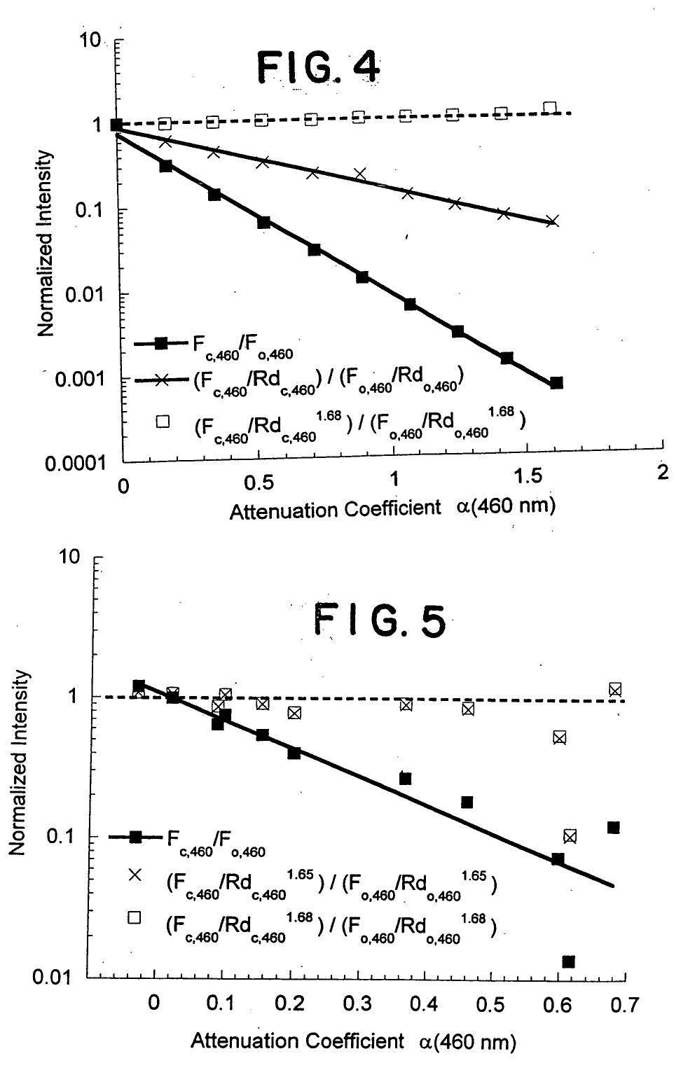

The results of the simulation are indicated in FIG. 4 and show that fluorescence emission decreases exponentially as the degree of the superficial blood contamination increases. The solid lines are curve fits to the simulated data. The dashed line represents the ideal outcome of the F-Rd combined numerical value [F_{c,460}/Rd_{c,460}^{h}]/[Fₒ₄₆₀/Rd_{o,460}^{h} at h=1.68. This agrees with the theoretical prediction in equation (2) above. The F-Rd combined numerical value [F_{o,460}/Rd_{c,460}^{h}]/[F_{o,460}^{h}/Rd_{o,460}^{h}] curve still decreases exponentially in FIG. 4, but at a slower rate as compared to the [F_{c,460}/F_{o,460}] curve in FIG. 4. This may explain why discrimination using the combined numerical value F₄₆₀/Rd₄₆₀ was more successful in separating normal and tumorous brain tissues in vivo than using F₄₆₀ alone. The effect of blood contamination was essentially completely removed in the combined numerical value F₄₆₀/Rd₄₆₀^{1.68} as evidenced by its corresponding curve in FIG. 4, which remains almost unchanged and horizontal.

To experimentally validate the relationship described above, fluorescence and diffuse reflectance spectra were measured from multiple tissue samples (see below) with varying degrees of blood contamination using a fiber optic based detection system 10 described above. Two excitation light sources were used: a nitrogen laser (337 nm, 20 ns laser pulse-width, 10 μJ/pulse at tissue surface, 20 Hz reception rate) for fluorescence and a broadband halogen light (2 mW at tissue surface) for diffuse reflectance. An integration time of two seconds was used for each spectral measurement, and three spectra, background, fluorescence, and diffuse reflectance, were sequentially acquired from each site.

Chicken breast muscle tissue was used as the tissue sample as it provides adequate fluorescence emission at 460 nm and its structure is relatively homogenous over a large area. Human blood drawn from a volunteer was diluted using phosphate buffered saline (PBS) and used as a surface absorbing media. The absorption spectrum of the diluted blood, μ_{a,blood+PBS}(λ), was measured using a spectrophotometer (Lambda 900, Perkin Elmer) and used to calculate the experimental value of h.

The fluorescence and diffuse reflectance spectra Fₒ(λ) and Rdₒ(λ), were first acquired from the investigated sample prior to introducing blood contamination. The optical probe was placed lightly in contact with the investigated tissue surface to avoid excessive compression of the tissue. A drop of the absorbing medium (diluted blood) was then applied to the surface of the investigated site, and blood contaminated tissue fluorescence and diffuse reflectance spectra, F_{c}(λ) and Rd_{c}(λ), were acquired. Each investigated site was used only once as the absorbing medium penetrated into the tissue and could not be completely removed.

All acquired optical spectra were first preprocessed to account for background signal and spectral variations introduced by the spectrometer. F_{c,460}/F_{o,460} was then calculated to determine the degree of fluorescence attenuation due to blood contamination at each site. In addition, the attenuation coefficient α at 460 nm was calculated from Rd_{c,460}/Rd_{o,460} at each site using equation (3) above. Finally, (F_{c}/Rd_{c}^{h})/(Fₒ/Rdₒ^{h}) was calculated at each site using the values of h determined by the measured μ_{a,blood+PBS}(λ) and by theory.

The results of the experimental study are shown in FIG. 5. Experimental normalized fluorescence intensities F_{c,460}/F_{o,460} and normalized [F_{c,460}/Rd_{c,460}^{h}]/[F_{o,460}/Rd_{o,460}^{h}] are shown for different levels of a at 460 nm. The solid line is fit to the experimental data. The dashed line represents the ideal outcome for h=1.65 or 1.68. The even distribution of the attenuation coefficient α, over the entire range suggests that different degrees of blood contamination were achieved. The one sample F_{c,460}/F_{o,460}>1 suggests that the fluorescence intensity at 460 nm emission from this particular site increased after the absorbing medium was applied. This error may be attributed to the reduced index-mismatch between the optical probe and the tissue due to the presence of the absorbing medium or the variation of laser pulse energy between the two measurements. The absorption spectrum of the diluted blood was very similar to that of oxyhemoglobin between 300 nm and 600 nm as expected. Based on the measured spectra, k=μ_{a,blood+PBS, 337}/μ_{a,blood+PBS, 460}=2.30 and h=(k+1)/2=1.65 were obtained. This is very close to the h number predicted by theory (i.e., 1.68). In FIG. 5, [F_{c,460}/Rd_{c,460}^{h}]/[F_{o,460}/Rd_{o, 460}^{h}] calculated using h=1.65 remains almost unchanged between α=0 and α=0.5, while F_{c,460}/F_{o,460} decreases exponentially as a increases. The experimental results deviate from the theoretical predictions only for α>0.6. It is believed that these deviations primarily result from poor signal to noise ratio in the spectra measured from sites with a high degree of blood contamination. When the theoretical number h was used, [F_{c,460} Rd_{c,460}^{h}]/F_{o,460}/Rd_{o,460}^{h}] also remains almost unchanged between α=0 and α=0.5. Nevertheless, this model is effective for α≦0.5 corresponding to a fluorescence attenuation at 337 nm excitation, 460 nm emission of approximately 85%. Typical fluorescence signal attenuation encountered in in vivo optical spectroscopy is less than 70%. This implies that this technique is useful in a clinical situation (e.g., brain tumor resection).

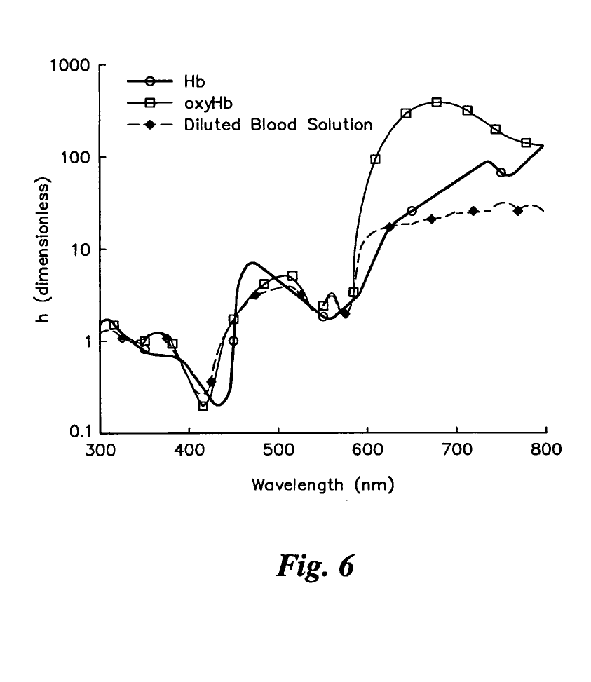

FIG. 6 depicts the variation in values for the absorption dependent coefficient h for hemoglobin (Hb), oxygenated (oxy-)hemoglobin and diluted blood solution as described above at different indicated emission wavelengths for an incident wavelength of about 330 nm (337 nm). Of the three, the diluted blood solution is of primary interest since it attempts to Q duplicate surface blood contamination. In the emission range of interest from about 300 nm to about 700 nm, the exponential value of “h” for diluted blood solution varies from a low of about 0.2 (0.22) to a high of about 25 (24.2). In the narrower emission range of about 300 nm to about 600 nm, the exponential value “h” varies from a low of about 0.2 to a high of about 12 (11.8). In the emission range of about 400 nm to about 500 nm “h” varies from a low of about 0.2 to a high of about 4 (3.8). The value h only exceeds 1 for emission wavelengths of about 440 nm or more. For excitation wavelengths above or below about 330 nm, the three curves, depicted in FIG. 6 can shift up or down (i.e. the value of h can increase or decrease at a given emission wavelength).

The above shows that combined optical spectroscopy, in particular a ratio of F_{c}(λₘ)/Rd_{c}(λₘ)^{(h(λx,λm)} where h is a mixed number, minimizes the effect of blood contamination thus alleviating the major obstacle towards the application of optical spectroscopy for surgical guidance. This relationship has been validated in a simulation as well as experimentally. It should be noted that h(λx, λm) can be predetermined using the absorption spectrum of whole blood. Hence, the combined optical spectrum numerical value, F_{c}(λₘ)/Rd_{c}(λₘ)^{h(λx,λm)}, instead of the fluorescence spectrum value F_{c}(λₘ) alone, should be used in the development of in vivo tissue discrimination algorithms for detection of tissue.

It will be further appreciated that the ratio F/Rd^{h} can be manipulated to provide equivalent results. For example, the ratio could be inverted and then rescaled by multiplication by a constant. Furthermore, the experimental coefficient of the autofluorescent return intensity (F) can be varied from unity (1) and that of the diffuse reflectance maintained at unity instead. Thus, F^{h1}/R where h1 is about 0.6 should give a similarly blood insensitive result. According to the invention, a ratio of fluorescence F and diffuse reflectance Rd intensities is taken with at least one of the intensities being an exponential power other than unity and zero. The power is selected to reduce the effect of blood attenuation on the combination of the two light intensities.

There is another convenient way of combining F and Rd spectra, which results in a combined spectrum numerical value at least essentially not affected by superficial blood contamination. As stated in the previous section, the fluorescence (autofluorescent) signal measured from a tissue sample with superficial blood contamination can be described as

F(λₘ)_{c}=F(λₘ)ₒ×exp(−μa(λₓ)×d)×exp(−μo(λₘ)×d) (5)

The ratio of the fluorescence signals at λₘ and λ_{ref} would yield

F(λₘ)_{c}/F(λ_{ref})_{c}=[F(λₘ)ₒ/F(λ_{ref})ₒ]×[exp(−μa(λₘ)×d)/exp(−μa(λ_{ref})×d)] (6)

where λₘ and λ_{ref} are arbitrary wavelengths. Applying the same spectral processing procedure to the diffuse reflectance spectra would yield

Rd(λₘ)_{c}/Rd(λ_{ref})_{c}=[Rd(λₘ)ₒ/Rd(λ_{ref})ₒ]×[exp(−2×μa(λₘ)×d)/exp(−2×μa(λ_{ref})×d)] (7)

Comparing Eqs. (6) and (7), it is clearly that the exponential terms, introduced by the superficial blood contamination, can be eliminated by taking take the ratio of [F(λₘ)_{c}/F(λ_{ref})_{c}] and Rd(λₘ)_{c}/Rd(λ_{ref})_{c}. That is:

[F(λₘ)_{c}/F(λ_{ref})_{c}]²/[Rd(λₘ)_{c}/Rd(λ_{ref})_{c}]=F(λₘ)ₒ/F(λ_{ref})ₒ]²/[Rd(λₘ)ₒ/Rd(λ_{ref})ₒ] (8)

Therefore, the combined F-Rd spectrum numerical value, [F(λₘ)_{c}/F(λ_{ref})_{c}]²/[Rd(λₘ)_{c}/Rd(λ_{ref})_{c}], is free from superficial blood contamination effects. More importantly, this combination removes the dependence of the fluorescence and diffuse reflectance spectra on excitation power. Hence, artifacts generated by the fluctuations of the excitation power among spectral acquisitions can be eliminated in spectral data analysis. The combined F-Rd spectrum numerical value on the left side of equation (8) can also be expressed as:

[F(λₘ)_{c}²/Rd(λₘ)_{c}][Rd(λ_{ref})_{c}/F(λ_{ref})_{c}²] (9)

and, alternatively, as:

[F(λₘ)_{c}²/Rd(λₘ)_{c}]/[F((λ_{ref})_{c}²/Rd(λ_{ref})_{c}] (10)

It will thus be appreciated that ratios of autofluorescent and diffuse reflectance intensities at specific wavelengths continue to be utilized to generate this combined F-Rd spectrum numerical value of the illuminated tissue area.

Tumor ablation using a Free Electron Laser (FEL) 90 (see FIG. 1) has also been investigated. FEL is believed to be an ideal tool for removing residual tumor mass at a brain tumor boundary because it provides wavelength tunability and high precision in terms of tissue ablation. The ablation of native (normal) and tumorous brain tissue with FEL pulses of various laser parameters (e.g. energy density) was examined. Autofluorescence emission and diffuse reflectance were measured at the ablation sites before and immediately after FEL ablation. With sufficient laser energy (e.g. 70 J/sq. cm.), both 3 μm and 6 μm FEL ablated brain tissue c cleanly. No sign of thermal damage (i.e. tissue whitening) was visually observed after ablation. More importantly, the autofluorescence and diffuse spectra of brain tissues within the ablation zones remained unchanged. In contrast. Ablation using FEL pulses with energy densities slightly above the ablation threshold caused significant amounts of thermal damage. This was n especially noticeable for gray matter. Significant increases in autofluorescence emission and diffuse reflectance were consistently measured from coagulated tissues after ablation. In some cases. Autofluorescence emission or diffuse reflectance from coagulated brain tissues were found to be three or four times greater than those measured from native brain tissues. It was further found that coagulated brain tissues have a much higher scattering coefficient compared to that of native brain tissues at any given wavelength in the visible light spectrum. Accordingly, FEL pulses with energy densities several times that of the ablation threshold, suggestedly at least three and preferably at least four times that of the ablation threshold (e.g., 70 J/sq.cm. or more at λ=6.4 μm) should be used to cleanly ablate the affected brain tissue without altering the spectral features of surrounding brain tissues by photocoagulation.

Referring back to FIG. 1, initially the FEL is guided manually by the surgeon in response to the tumorous/non-tumorous output of the system 10. However, it is currently envisioned that the FEL and system would be combined to use a single probe with the FEL operation being automatically controlled by the system controller.

The contents of U.S. Patent Application No. 60/193,491 and Ser. No. 09/545,425 are incorporated by reference herein in all their entireties.

It should further be appreciated that the 460 and 625 nm optimal spectral values were for the described equipment operation and calibration and that other equipment arrangement, operations and/or calibrations may yield somewhat different spectral value peaks that will have to be determined empirically preferably by in vitro testing. It is still expected that the optimal combined spectral values will lie within a range of about ±20 around 460 nm and 625 nm. For the mixed experimental combined spectral values λₘ and λ_{ref}, the measurement wavelength λₘ should suggestedly remain in the 400 to 600 nm range while the reference wavelength λ_{ref} is suggestedly selected from the 600 to 800 nm range, more specifically the 650 to 700 nm range. The wavelengths λₘ, λ_{ref} should be selected to optimize the discrimination results for the particular equipment/procedure/calibration utilized.

It will be appreciated by those skilled in the art that changes could be made to the embodiments described above without departing from the broad inventive concept thereof. For example, while conventional lasers producing monochromatic light are currently used, so-called broad band lasers are under development and their possible use in the present invention is considered to be within the scope of the invention. It is understood, therefore, that this invention is not limited to the particular embodiments disclosed, but it is intended to cover modifications within the spirit and scope of the present invention as defined by the appended claims.

I/we claim:

1. A method for optically identifying human tissue type comprising the steps of:
illuminating a surface area of tissue to be identified with a source of white light and gathering diffuse reflectance light returned from the illuminated tissue area;
illuminating the surface area of the tissue to be identified with a source of monochromatic light and gathering autofluorescent light emitted by the tissue area in response to the monochromatic light illumination, the illumination and gathering of the diffuse reflectance light and the autofluorescent light occurring in either order;
generating a numerical value based at least in part upon a first ratio of a value of intensity of the diffuse reflectance light gathered from the illuminated tissue area and a value of intensity of the autofluorescent light gathered from the illuminated tissue area; and
using the numerical value to identify the type of tissue of the area illuminated.

2. The method according to claim 1 wherein the numerical value is used to distinguish between tumorous and non-tumorous tissue

3. The method according to claim 2 wherein the numerical value is used to distinguish between tumorous brain tissue and normal brain tissue.

4. The method according to claim 1 wherein the monochromatic light illuminating step comprises illuminating the tissue surface with coherent light at a wavelength of about 330 nm.

5. The method according to claim 1 wherein the first ratio is generated using the value of the intensity of the diffuse reflectance light at one wavelength and the value of the intensity of the autofluorescent light at the one wavelength.

6. The method according to claim 5 wherein the one wavelength of the intensity of the diffuse reflectance light and the intensity of the autofluorescent light used in the first ratio is selected from a range of between 300 nm and 700 nm.

7. The method according to claim 5 wherein the one wavelength of the intensity of the diffuse reflectance light and the intensity of the autofluorescent light used in the first ratio is selected from a range of between 400 nm and 600 nm.

8. The method according to claim 5 wherein the one wavelength of the intensity of the diffuse reflectance light and the intensity of the autofluorescent light used in the first ratio is selected from a range of 450 nm. to 470 nm.

9. The method according to claim 5 wherein the one wavelength of the intensity of the diffuse reflectance light and the intensity of the autofluorescent light used in the first ratio is about 460 nm.

10. The method according to claim 5 further comprising the step of identifying a second value of intensity of the diffuse reflectance light from the illuminated area at a second wavelength different from the one wavelength of the diffuse reflectance intensity used in the first ratio and wherein the using step further comprises using the second intensity value with the first ratio to identity the type of tissue illuminated.

11. The method according to claim 10 further comprising the steps of identifying a second value of intensity of the autofluorescent light from the illuminated area at the second wavelength, generating a second ratio including the second value of intensity of the diffuse reflectance light with the second value of intensity of the autofluoresecent light and combining the first and second ratios to provide the numerical value.

12. The method according to claim 1 wherein the value of the intensity of the diffuse reflectance light used to generate the first ratio is an other than zero exponential value of the intensity of the diffuse reflectance light other than zero and different from an other than zero exponential value of the intensity of autofluorescent light used to generate the first ratio.

13. The method according to claim 12 wherein the first ratio is generated using the value of the intensity of the diffuse reflectance light at only one wavelength and the value of the intensity of the autofluorescent light at the one wavelength.

14. The method according to claim 13 wherein the one wavelength is selected from a range of between 400 nm. to 600 nm.

15. The method according to claim 14 wherein the one wavelength is about 460 nm.

16. The method according to claim 13 further comprising the step of identifying a second value of intensity of the diffuse reflectance light from the illuminated area at a second wavelength different from the one wavelength of the diffuse reflectance intensity used in the first ratio and wherein the using step further comprises using the second intensity value with the first ratio to identity the type of tissue illuminated.

17. The method according to claim 16 wherein the intensity of the diffuse reflectance light used to generate the first ratio has a blood absorption coefficient dependent exponential value (h) greater than an exponential value of the intensity of the autofluorescent light used to generate the first ratio.

18. The method according to claim 17 wherein the exponential value of the diffuse reflectance light used to generate the first ratio is about twenty five times or less than the exponential value of the autofluorescent light used to generate the first ratio.

19. The method according to claim 17 wherein the exponential value of the diffuse reflectance light used to generate the first ratio is about twelve times or less than the exponential value of the autofluorescent light used to generate the first ratio.

20. The method according to claim 17 wherein the exponential value of the diffuse reflectance light used to generate the first ratio is about four times or less than the exponential value of the autofluorescent light used to generate the first ratio.

21. The method according to claim 17 wherein the exponential value of the diffuse reflectance light used to generate the first ratio is less than two times of the exponential value of the autofluorescent light used to generate the first ratio.

22. The method according to claim 17 wherein the exponential value of the diffuse reflectance light used to generate the first ratio is between about one and two-thirds the exponential value of the autofluorescent light used to generate the first ratio.

23. The method according to claim 12 wherein the exponential value of the diffuse reflectance light used to generate the first ratio is greater than the exponential value of the autofluorescent light used to generate the first ratio.

24. The method according to claim 23 wherein the exponential value of the diffuse reflectance light used to generate the first ratio is about twenty-five times or less than the exponential value of the autofluorescent light used to generate the first ratio.

25. The method according to claim 23 wherein the exponential value of the diffuse reflectance light used to generate the first ratio is about twelve times or less than the exponential value of the autofluorescent light used to generate the first ratio.

26. The method according to claim 23 wherein the exponential value of the diffuse reflectance light used to generate the first ratio is less than two times of the exponential value of the autofluorescent light used to generate the first ratio.

27. The method according to claim 23 wherein the exponential value of the diffuse reflectance light used to generate the first ratio is about one and two-thirds the exponential value of the autofluorescent light used to generate the first ratio.

28. The method according to claim 1 wherein an exponential value of the intensity of the diffuse reflectance light is used to generate the first ratio and is different from zero and from an exponential value of the intensity of the autofluorescent light used to generate the first ratio.

29. The method according to claim 1 wherein the first ratio is generated using at least one exponential value other than unity and other than zero of at least one of the intensity of the diffuse reflectance light and the intensity of the autofluorescent light, the at least one exponential value being selected to reduce variation in the numerical value caused by the presence of blood contamination of the surface area being illuminated.

30. The method according to claim 1 further comprising the step of identifying a second value of intensity of the diffuse reflectance light from the illuminated area at a second wavelength different from any wavelength of the diffuse reflectance intensity used in the first ratio and wherein the using step further comprises using the second intensity value with the first ratio to identity the type of tissue illuminated.

31. The method according to claim 30 further comprising the steps of identifying a second value of intensity of the autofluorescent light from the illuminated area at the second wavelength and generating a second ratio including the second value of intensity of the diffuse reflectance light with the second value of the autofluorescent light and using the second ratio with the first ratio to generate the numerical value.

32. The method according to claim 30 wherein the one wavelength is selected from a range of between 300 nm 700 nm.

33. The method according to claim 30 wherein the one wavelength is selected from a range of between 300 nm. to 600 nm.

34. The method according to claim 30 wherein the one wavelength is selected from a range of between 400 nm to 600 nm.

35. The method according to claim 30 wherein the one wavelength is about 460 nm.

36. The method according to claim 1 wherein the generating step further includes:
generating a second ratio of a second value of intensity of the diffuse reflectance light from the illuminated area at a second wavelength different from any wavelength of the diffuse reflectance light intensity used in the first ratio and of a second value of intensity of the autofluorescent light from the illuminated area at the second wavelength; and
combining the second ratio with the first ratio to generate the numerical value of the illuminated area.

37. The method according to claim 36 wherein an exponential value other than zero of the intensity of the diffuse reflectance light is used to generate at least the first ratio and is different from an exponential value other than zero of the intensity of the autofluorescent light used to generate the first ratio.

38. The method according to claim 37 wherein an exponential value other than zero of the intensity of the diffuse reflectance light is used to generate the second ratio and is different from an exponential value other than zero of the intensity of the autofluorescent light used to generate the second ratio.

39. The method according to claim 38 wherein the exponential value of the intensity of the diffuse reflectance light used to generate the first ratio is the same as the exponential value of the diffuse reflectance light used to generate the second ratio.

40. The method according to claim 38 wherein the exponential value of the autofluorescent light used to generate the first ratio is the same as the exponential value of the autofluorescent light used to generate the second ratio.

41. The method according to claim 38 wherein the exponential value of the autofluorescent light used to generate the first ratio is twice the exponential value of the diffuse reflectance light used to generate the first ratio.

42. The method according to claim 36 wherein the exponential values of the autofluorescent and diffuse reflectance intensities used in the first and second ratios are independent of each of the first and second wavelengths.

43. The method according to claim 12 wherein the exponential value of the diffuse reflectance light is related, at least in part, to the first wavelength.

44. The method according to claim 12 wherein the exponential value of the diffuse reflectance light is related, at least in part, to a wavelength of the monochromatic light.
